(19) United States
(12) Patent Application Publication
(10) Pub. No.: US 2026/0144844 A1
(43) Pub. Date: May 28, 2026

(54) PHARMACEUTICAL COMPOSITIONS FEATURING MIXTURES OF PEPTOIDS WITH AND WITHOUT TERMINAL ALIPHATIC MOIETIES

(71) Applicant: Maxwell Biosciences, Inc., Austin, TX (US)

(72) Inventors: Annelise E. Barron, Woodside, CA (US); John A. Fortkort, Austin, TX (US)

(21) Appl. No.: 19/103,497

(22) PCT Filed: Aug. 14, 2023
(86) PCT No.: PCT/US2023/072150

§ 371 Date: Feb. 12, 2025

Related U.S. Application Data
(60) Provisional application No. 63/371,524, filed on Aug. 16, 2022.

(51) Int. Cl. A61K 38/08; A61K 38/10

(52) U.S. Cl. CPC A61K 38/08; A61K 38/10

(57) ABSTRACT

Compositions are provided which feature mixtures of at least a first and second peptoid. The second peptoid has a terminal aliphatic moiety, while the first peptoid lacks a terminal aliphatic moiety. These compositions have various end uses, including the treatment of various cancers and tumors and the treatment of bacterial, fungal, microbial or parasitic infections.

### CROSS REFERENCE TO RELATED APPLICATION

This application claims the benefit under 35 U.S.C. § 119 (e) of U.S. Provisional Application No. 63/371,524, filed Aug. 16, 2022, the contents of which are incorporated by reference herein in their entirety.

### FIELD OF THE DISCLOSURE

The present disclosure relates generally to peptoids, and more particularly to mixtures of peptoids featuring at least a first peptoid containing an alkylated tail, and a second peptoid lacking such a tail.

### BACKGROUND OF THE DISCLOSURE

Peptoids (or poly-N-substituted glycines) are a class of peptidomimetics whose side chains are appended to the nitrogen atom of the peptide backbone, rather than to the α-carbons as they are in amino acids (see FIG. 1). This modification renders peptoids more resistant to proteolytic degradation than their peptide counterparts.

Peptoids have found various uses as therapeutic agents. For example, commonly assigned U.S. Pat. No. 8,445,632 (Barron et al.), entitled “Selective poly-N-substituted glycine antibiotics”, discloses a family of peptoids that are active against bacterial infections. U.S. Pat. No. 9,938,321 (Kirshenbaum et al.), U.S. Pat. No. 9,315,548 (Kirshenbaum et al.) and U.S. Pat. No. 8,828,413 (Kirshenbaum et al.) disclose cyclic peptoids that exhibit antimicrobial and antimalarial activity.

Various attempts have been made in the art to improve certain characteristics of peptoids. For example, commonly assigned PCT/US20/30890 (Molchanova et al.) disclose a class of halogenated peptoids. The halogenation was found, in some instances, to enhance the self-assembly and antimicrobial potency of these materials compared to their nonhalogenated analogs.

### SUMMARY OF THE DISCLOSURE

In one aspect, a composition is provided which comprises a first peptoid which is a poly-N-substituted glycine compound of a formula

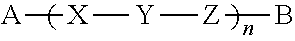

wherein
A is a terminal N-alkyl substituted glycine residue,
n is an integer,
B is selected from the group consisting of NH₂, one and two N-substituted glycine residues, and wherein said one and two N-substituted glycine residues have N-substituents which are independently selected from natural α-amino acid side chain moieties, isomers and carbon homologs thereof, and
X, Y and Z are independently selected from the group consisting of N-substituted glycine residues, wherein said N-substituents are independently selected from the group consisting of natural α-amino acid side chain moieties, isomers and carbon homologs thereof, and proline residues; and
a second peptoid which is distinct from said first peptoid and which is a poly-N-substituted glycine compound of a formula

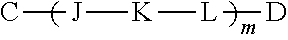

wherein
C is a terminal N-alkyl substituted glycine residue of the formula

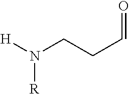

R is an alkyl group,
m is an integer,
D is selected from the group consisting of NH₂, one and two N-substituted glycine residues, and wherein said one and two N-substituted glycine residues have N-substituents which are independently selected from natural α-amino acid side chain moieties, isomers and carbon homologs thereof, and
J, K and L are independently selected from the group consisting of N-substituted glycine residues, wherein said N-substituents are independently selected from the group consisting of natural α-amino acid side chain moieties, isomers and carbon homologs thereof, and proline residues.

In another aspect, a method is provided for treating a subject for cancer. The method comprises diagnosing the subject as having cancer; and treating the subject with a pharmaceutically acceptable amount of the foregoing composition.

In a further aspect, a method is provided for treating a subject for a viral infection. The method comprises diagnosing the subject as having a viral infection; and treating the subject with a pharmaceutically acceptable amount of the foregoing composition.

In another aspect, a method is provided for treating a subject for a parasitic infection. The method comprises diagnosing the subject as having a fungal infection; and treating the subject with a pharmaceutically acceptable amount of the foregoing composition.

In still another aspect, a method is provided for treating a subject for a bacterial infection. The method comprises diagnosing the subject as having a bacterial infection; and treating the subject with a pharmaceutically acceptable amount of the foregoing composition.

In still another aspect, a method is provided for treating a subject for a microbial infection. The method comprises diagnosing the subject as having a bacterial infection; and treating the subject with a pharmaceutically acceptable amount of the foregoing composition.

In yet another aspect, a method is provided for treating a subject for a parasitic infection. The method comprises diagnosing the subject as having a parasitic infection; and treating the subject with a pharmaceutically acceptable amount of the foregoing composition.

### BRIEF DESCRIPTION OF THE DRAWINGS

FIG. 1 is a diagram illustrating the structural differences between peptides and peptoids.

FIG. 2 is an illustration of the chemical structure of various monomers which may be utilized in making the peptoids described herein.

FIG. 3 is a listing of some of the peptoids which may be utilized in the compositions and methodologies disclosed herein.

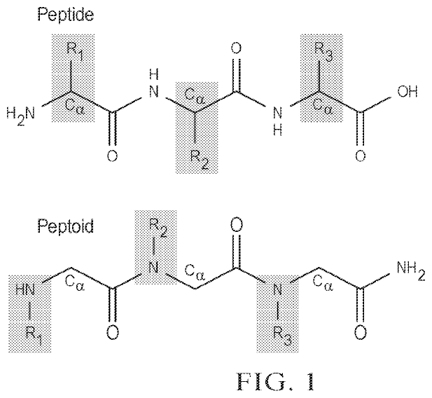

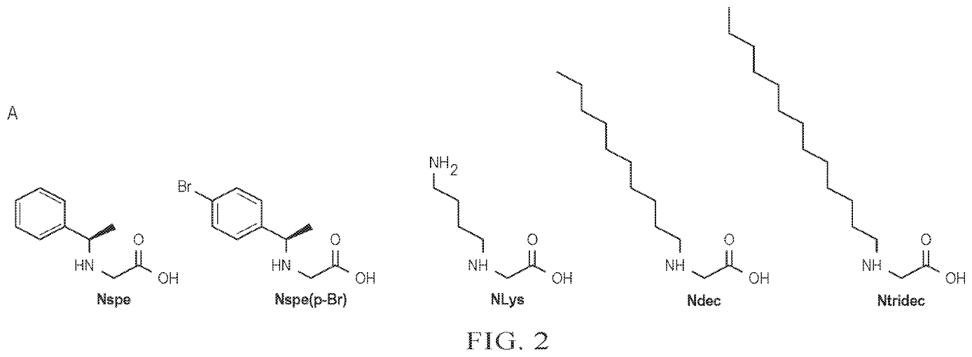

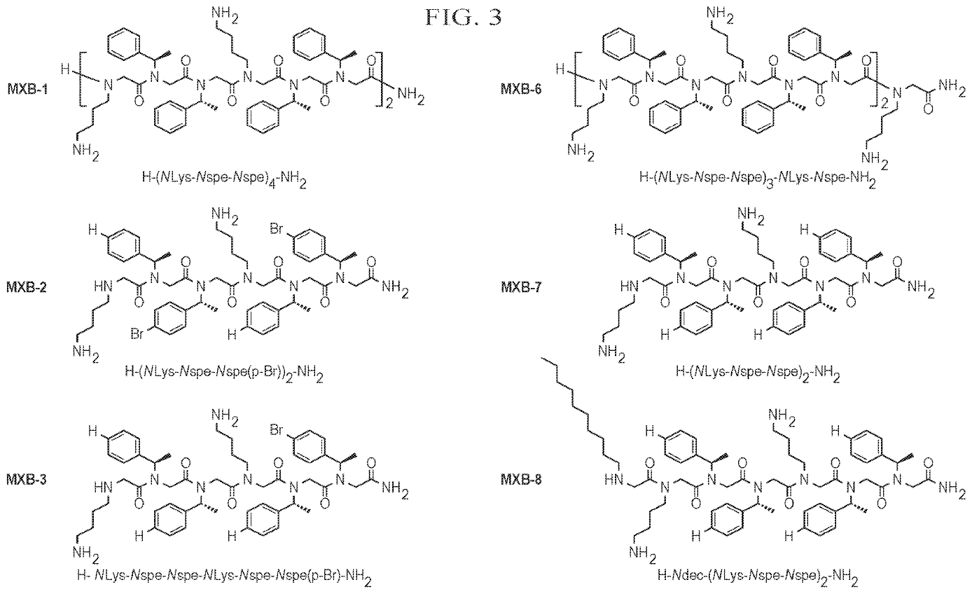

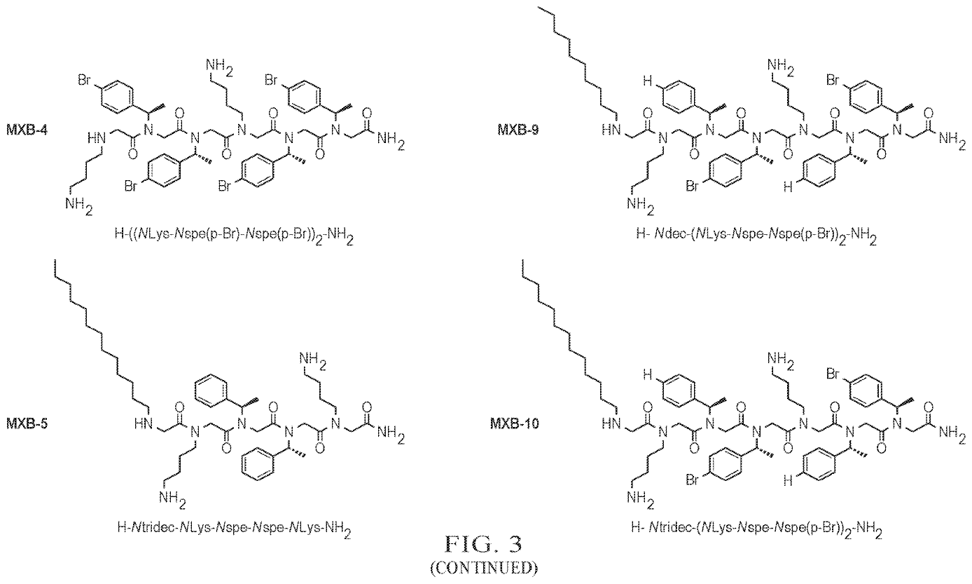

### DETAILED DESCRIPTION

It has now been found that the efficacy of peptoids in some applications may be enhanced by using two or more diverse peptoids in combination with each other. In a preferred embodiment, for example, pharmaceutical compositions are provided which comprise at least a first peptoid which contains an alkylated tail, and at least a second peptoid which lacks such a tail. In some applications, mixtures of peptoids of this type are found to exhibit a synergistic effect. Such applications may include, but are not limited to, the treatment of various types of cancers and infections, the latter of which include viral infections, fungal infections, bacterial infections, microbial infections and parasitic infections.

In one exemplary embodiment of the methodologies disclosed herein, a patient is diagnosed as having a cancer or tumor. A therapeutic amount of a composition of the foregoing type is then administered to the patient.

Various peptoids may be utilized in the compositions and methodologies disclosed herein. These include the peptoids having the designations and structures depicted in TABLE 1 below. It is to be noted that the letter precursors TM and MXB may be used interchangeably in the designations of these peptoids (that is, for example, MXB016 and TM016 refer to the same peptoid).

TABLE 1
Designations and Structures of Peptoids

| | | Chemical | |
|---|---|---|---|
| Peptoid | Structure | Formula | M.W. |
| MXB001 | H-(NLys-Nspe-Nspe)₄-NH₂ | C₁₀₄H₁₃₉N₁₇O₁₂ | 1819.36 |
| MXB002 | H-(NLys-Nspe-Nspe(p-Br))₂-NH₂ | C₅₂H₆₉N₉O₆ | 1075.99 |
| MXB003 | H-NLys-Nspe-Nspe-NLys-Nspe-Nspe(p-Br)-NH₂ | | |
| MXB004 | H-((NLys-Nspe(p-Br)-Nspe(p-Br))₂-NH₂ | C₅₂H₆₇Br₄N₉O₆ | 1233.78 |
| MXB005 | H-Ntridec-NLys-Nspe-Nspe-NLys-NH₂ | C₄₇H₇₈N₈O₅ | 835.19 |
| MXB007 | H-(NLys-Nspe-Nspe)₃-NLys-Nspe-NH₂ | | |
| MXB008 | H-(NLys-Nspe-Nspe)₂-NH₂ | C₆₄H₉₄N₁₀O₇ | 1115.52 |
| MXB009 | H-Ndec-(NLys-Nspe-Nspe)₂-NH₂ | C₆₄H₉₂N₁₀O₇ | 1273.31 |
| MXB010 | H-Ndec-(NLys-Nspe-Nspe(p-Br))₂-NH₂ | | |
| MXB011 | H-Ntridec-(NLys-Nspe-Nspe(p-Br))₂-NH₂ | C₁₁₀H₁₅₁N₁₉O₁₃ | 1947.54 |
| MXB012 | H-(NLys-Nspe-Nspe)₄-NLys-NH₂ | C₅₈H₈₁Br₂N₁₁O₇ | 1204.16 |
| MXB013 | H-(NLys-Nspe-Nspe(p-Br))₂-NLys-NH₂ | | |
| MXB014 | H-NLys-Nspe-Nspe-NLys-Nspe-Nspe(p-Br)-NLys-NH₂ | C₅₈H₇₉Br₄N₁₁O₇ | 1361.96 |
| MXB015 | H-(NLys-Nspe(p-Br)-Nspe(p-Br))₂-NLys-NH₂ | C₅₃H₉₀N₁₀O₆ | 962.70 |
| MXB016 | H-Ntridec-NLys-Nspe-Nspe-NLys-NLys-NH₂ | | |
| MXB017 | H-(NLys-Nspe-Nspe)₃-NLys-Nspe-NLys-NH₂ | | |
| MXB018 | H-(NLys-Nspe-Nspe)₂-NLys-NH₂ | | |
| MXB019 | H-Ndec-(NLys-Nspe-Nspe)₂-NLys-NH₂ | C₇₀H₁₀₄Br₂N₁₂O₈ | 1401.49 |
| MXB020 | H-Ndec-(NLys-Nspe-Nspe(p-Br))₂-NLys-NH₂ | | 1440 |
| MXB021 | H-Ntridec-(NLys-Nspe-Nspe(p-Br))₂-NLys-NH₂ | | |
| MXB022 | H-(NLys-Nssb-Nssb)₄-NH₂ | | |

In preferred embodiments, the mixtures of peptoids disclosed herein feature at least a first and second peptoid. The first peptoid is a poly-N-substituted glycine compound of a formula

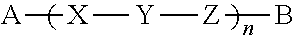

wherein
A is a terminal N-alkyl substituted glycine residue,
n is an integer,
B is selected from the group consisting of NH₂, one and two N-substituted glycine residues, and wherein said one and two N-substituted glycine residues have N-substituents which are independently selected from natural α-amino acid side chain moieties, isomers and carbon homologs thereof, and
X, Y and Z are independently selected from the group consisting of N-substituted glycine residues, wherein said N-substituents are independently selected from the group consisting of natural α-amino acid side chain moieties, isomers and carbon homologs thereof, and proline residues.

The second peptoid is distinct from the first peptoid and which is a poly-N-substituted glycine compound of a formula

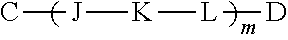

wherein
C is a terminal N-alkyl substituted glycine residue of the formula

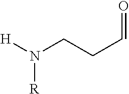

R is an alkyl group,
m is an integer,
D is selected from the group consisting of NH₂, one and two N-substituted glycine residues, and wherein said one and two N-substituted glycine residues have N-substituents which are independently selected from natural α-amino acid side chain moieties, isomers and carbon homologs thereof, and
J, K and L are independently selected from the group consisting of N-substituted glycine residues, wherein said N-substituents are independently selected from the group consisting of natural α-amino acid side chain moieties, isomers and carbon homologs thereof, and proline residues.

As previously noted, in preferred embodiments, one of the peptoids in the compositions described herein may be alkylated, and preferably has terminal alkylation. Here, alkylation (and especially terminal alkylation) with a C10 or C13 tail is especially preferred. It has been found that such terminal alkylation may enhance the efficacy of the peptoid in some applications. Thus, in the foregoing formula, R may be selected from about C4 to about C20 linear, branched and cyclic alkyl moieties. R preferably contains at least 8 carbon atoms, more preferably contains at least 10 carbon atoms, and most preferably contains at least 12 carbon atoms. Embodiments where R is a decyl or tridecyl moiety are especially preferred.

As previously noted, various peptoids and oligomers of N-substituted glycines may be utilized in the compositions and methodologies disclosed herein. In addition to the peptoids set forth in TABLE 1, these may include the peptoids described in U.S. Pat. No. 8,445,632 (Barron et al.), which is incorporated herein by reference in its entirety. In some embodiments, these may also include the peptoids disclosed in U.S. Pat. No. 9,938,321 (Kirshenbaum et al.), U.S. Pat. No. 9,315,548 (Kirshenbaum et al.) and U.S. Pat. No. 8,828,413 (Kirshenbaum et al.), all of which are incorporated herein by reference in their entirety.

It will further be appreciated that various salts or precursors of the foregoing peptoids may be utilized in the compositions and methodologies disclosed herein. As an example of the latter, these compositions and methodologies may contain one or more precursors of the general formula A-P, where P is one of the foregoing peptoids (or a salt thereof) and A is a moiety which acts as a leaving group to generate P when the precursor undergoes an in vivo reaction (such as, for example, proteolytic degradation).

Various halogenated peptoids and halogenated oligomers of N-substituted glycines (and salts thereof) may also be utilized in the compositions and methodologies disclosed herein. These include, without limitation, various halogenated analogs of the foregoing peptoids and oligomers of N-substituted glycines. These halogenated compositions may be halogenated in various ways. For example, these compounds may include any number of halogen substitutions with the same or different halogens. In particular, these compounds may include one or more fluoro-, chloro-, bromo- or iodo-substitutions, and may include substitution with two or more distinct halogens. However, the use of one or two bromo- or chloro-substitutions is preferred in many applications. Moreover, while the peptoids described herein may be halogenated at various locations, para halogenation on the peptoids containing aryl rings is especially preferred in many applications, although ortho- and meta-substitution, or even perhalogentation, may be useful in some applications.

The pharmaceutical compositions utilized in the systems and methodologies disclosed herein may utilize one or more active ingredients which may be dissolved, suspended or disposed in various media. Such media may include, for example, various liquid, solid or multistate media such as, for example, emulsions, gels or creams. Such media may include liquid media, which may be hydrophobic or may comprise one or more triglycerides or oils. Such media may include, but is not limited to, vegetable oils, fish oils, animal fats, hydrogenated vegetable oils, partially hydrogenated vegetable oils, synthetic triglycerides, modified triglycerides, fractionated triglycerides, and mixtures thereof. Triglycerides used in these pharmaceutical compositions may include those selected from the group consisting of almond oil; babassu oil; borage oil; blackcurrant seed oil; black seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; and mixtures thereof. The use of coconut oil is especially preferred.

The pharmaceutical compositions disclosed herein may be applied in various manners. Thus, for example, these compositions may be applied as oral, transdermal, transmucosal, intravenous or injected treatments, or via cell-based drug delivery systems. Moreover, these compositions may be applied in a single dose, multi-dose or controlled release fashion.

The pharmaceutical compositions disclosed herein may be manufactured as tablets, liquids, gels, foams, ointments or powders. In some embodiments, these compositions may be applied as microparticles or nanoparticles.

Various types of cancer may be treated with the compositions and methodologies disclosed herein. These include, without limitation, breast cancer, lung cancer (including small cell lung cancer), colorectal cancer, skin cancer, multiple myeloma, prostrate cancer, bone cancer, brain tumors, carcinoid tumors, cervical cancer, ductal carcinoma, endometrial cancer, esophageal cancer, gastric cancer, gastrointestinal stromal tumor (gist), HER2-positive breast cancer, islet cell tumors, juvenile polyposis syndrome, kidney cancer, laryngeal cancer, leukemia (including acute lymphoblastic leukemia, acute lymphocytic (all), acute myeloid (aml), chronic lymphocytic (cll), chronic myeloid (cml), and child and adult lukemia), liver cancer, lobular carcinoma, lymphoma (including both Hodgkin's and non-Hodgkin's), malignant glioma, melanoma, meningioma, myelodysplastic syndrome (mds), nasopharyngeal cancer, neuroendocrine tumors, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, polycythemia vera, renal cell carcinoma, retinoblastoma, salivary gland cancer, sarcoma, sarcoma-kaposi, skin cancer, small intestine cancer, stomach cancer, testicular cancer, thymoma, thyroid cancer, uterine (endometrial) cancer, vaginal cancer, and Wilms' tumors.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims. For convenience, some features of the claimed invention may be set forth separately in specific dependent or independent claims. However, it is to be understood that these features may be combined in various combinations and subcombinations without departing from the scope of the present disclosure. By way of example and not of limitation, the limitations of two or more dependent claims may be combined with each other without departing from the scope of the present disclosure.

What is claimed is:

A1. A composition, comprising:
a first peptoid which is a poly-N-substituted glycine compound of a formula 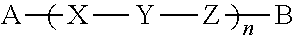
wherein
A is a terminal N-alkyl substituted glycine residue,
n is an integer,
B is selected from the group consisting of NH₂, one and two N-substituted glycine residues, and wherein said one and two N-substituted glycine residues have N-substituents which are independently selected from natural α-amino acid side chain moieties, isomers and carbon homologs thereof, and
X, Y and Z are independently selected from the group consisting of N-substituted glycine residues, wherein said N-substituents are independently selected from the group consisting of natural α-amino acid side chain moieties, isomers and carbon homologs thereof, and proline residues; and
a second peptoid which is distinct from said first peptoid and which is a poly-N-substituted glycine compound of a formula 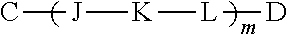
wherein
C is a terminal N-alkyl substituted glycine residue of the formula 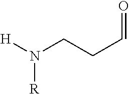
R is an alkyl group,
m is an integer,
D is selected from the group consisting of NH₂, one and two N-substituted glycine residues, and wherein said one and two N-substituted glycine residues have N-substituents which are independently selected from natural α-amino acid side chain moieties, isomers and carbon homologs thereof, and
J, K and L are independently selected from the group consisting of N-substituted glycine residues, wherein said N-substituents are independently selected from the group consisting of natural α-amino acid side chain moieties, isomers and carbon homologs thereof, and proline residues.

A2. The composition of claim A1, wherein R contains at least 8 carbon atoms.

A3. The composition of claim A1, wherein R contains at least 10 carbon atoms.

A4. The composition of claim A1, wherein R contains at least 12 carbon atoms.

A5. The composition of claim A1, wherein R is a decyl moiety.

A6. The composition of claim A1, wherein R is a tridecyl moiety.

A7. The composition of claim A1, wherein R is selected from about C4 to about C20 linear, branched and cyclic alkyl moieties.

A8. The composition of claim A1, wherein at least one of m and n have values within the range of 1-3.

A9. The composition of claim A1, wherein both m and n have values within the range of 1-3.

A10. The composition of claim A1, wherein at least one of said X, Y and Z residues is N_{Lys} and at least one said N-substituent is chiral.

A11. The composition of claim A1, wherein at least one of Y and Z are proline residues.

A12. The composition of claim A1, wherein Y and Z are proline residues.

A13. The composition of claim A1, wherein at least one of said J, K and L residues is N_{Lys} and at least one said N-substituent is chiral.

A14. The composition of claim A1, wherein at least one of K and L are proline residues.

A15. The composition of claim A1, wherein K and L are proline residues.

A16. The composition of claim A1, wherein A is a terminal N-alkyl substituted glycine residue, wherein said alkyl substituent selected from the group consisting of C6 to about C18 linear alkyl moieties, wherein B is NH₂, and wherein n is 1 or 2.

A17. The composition of claim A1, wherein A is a terminal N-alkyl substituted glycine residue, said alkyl substituent selected from about C6 to about C18 linear alkyl moieties; wherein B is an NLys residue; and wherein n is 1.

A18. The composition of claim A1, wherein J is a terminal N-alkyl substituted glycine residue, wherein said alkyl substituent selected from the group consisting of C6 to about C18 linear alkyl moieties, wherein D is NH₂, and wherein m is 1 or 2.

A19. The composition of claim A1, wherein J is a terminal N-alkyl substituted glycine residue, said alkyl substituent selected from about C6 to about C18 linear alkyl moieties; wherein D is an N_{Lys} residue; and wherein m is 1.

A20. The composition of claim A1, wherein the first peptoid is a hexamer.

A21. The composition of claim A1, wherein the first peptoid is a dodecamer.

A22. The composition of claim A1, wherein the first peptoid is a hexamer.

A23. The composition of claim A1, wherein the first peptoid is a dodecamer.

A24. The composition of claim A1, wherein the second peptoid is a hexamer.

A25. The composition of claim A1, wherein the second peptoid is a dodecamer.

A26. The composition of claims A1-A25, wherein at least one of A, B, X, Y and Z contains a halogen-bearing moiety.

A27. The composition of claim A26, wherein said halogen-bearing moiety contains a halogen-substituted aryl moiety.

A28. The composition of claim A26, wherein said halogen-bearing moiety contains a chloro-substituted aryl moiety.

A29. The composition of claim A28, wherein said halogen-bearing moiety contains a bromo-substituted aryl moiety.

A30. The composition of claim A26, wherein said halogen-bearing moiety contains an iodo-substituted aryl moiety.

A31. The composition of claim A26, wherein each mer in the hexamer contains a halogen-substituted aryl moiety.

A32. The composition of claim A26, wherein some of the mers in the hexamer contain a halogen-substituted aryl moiety, and wherein some of the mers in the hexamer contain a halogen-free aryl moiety.

A33. The composition of claim A26, wherein exactly one of the mers in the hexamer contains a halogen-substituted aryl moiety.

A34. The composition of claim A26, wherein each mer in the hexamer contains a halogen-substituted aryl moiety.

A35. The composition of claim A26, wherein some of the mers in the hexamer contain a halogen-substituted aryl moiety, and wherein some of the mers in the hexamer contain a halogen-free aryl moiety.

A36. The composition of claim A26, wherein only the first and last mers in the hexamer contain a halogen-substituted aryl moiety.

A37. The composition of claim A26, wherein at least two of A, B, X, Y and Z contain a halogen-bearing moiety.

A38. The composition of claim A26, wherein all of A, B, X, Y and Z contain a halogen-bearing moiety.

A39. The composition of claims A1-A25, wherein at least one of C, D, J, L and M contains a halogen-bearing moiety.

A40. The composition of claim A39, wherein said halogen-bearing moiety contains a halogen-substituted aryl moiety.

A41. The composition of claim A39, wherein said halogen-bearing moiety contains a chloro-substituted aryl moiety.

A42. The composition of claim A41, wherein said halogen-bearing moiety contains a bromo-substituted aryl moiety.

A43. The composition of claim A39, wherein said halogen-bearing moiety contains an iodo-substituted aryl moiety.

A44. The composition of claim A39, wherein each mer in the hexamer contains a halogen-substituted aryl moiety.

A45. The composition of claim A39, wherein some of the mers in the hexamer contain a halogen-substituted aryl moiety, and wherein some of the mers in the hexamer contain a halogen-free aryl moiety.

A46. The composition of claim A39, wherein exactly one of the mers in the hexamer contains a halogen-substituted aryl moiety.

A47. The composition of claim A39, wherein each mer in the hexamer contains a halogen-substituted aryl moiety.

A48. The composition of claim A39, wherein some of the mers in the hexamer contain a halogen-substituted aryl moiety, and wherein some of the mers in the hexamer contain a halogen-free aryl moiety.

A49. The composition of claim A39, wherein only the first and last mers in the hexamer contain a halogen-substituted aryl moiety.

A50. The composition of claim A39, wherein at least two of C, D, J, L and M contain a halogen-bearing moiety.

A51. The composition of claim A39, wherein all of C, D, J, L and M contain a halogen-bearing moiety.

A52. The composition of claims A1-A51, wherein said pharmaceutical composition contains a pharmaceutically acceptable salt of at least one of the first and second peptoids.

A53. The composition of claims A1-A52, wherein said pharmaceutical composition contains pharmaceutically acceptable salt of the first and second peptoids.

A54. The composition of claim A1-A52, wherein said first peptoid is H-(NLys-Nspe-Nspe) ₄-NH₂.

A55. The composition of claim A1-A52, wherein said first peptoid is Cy5.5-Ahx-(NLys-Nspe-Nspe) ₄-NH₂.

A56. The composition of claim A1-A52, wherein said first peptoid is H-(NLys-Nspe-Nspe (p-Br))₂-NH₂.

A57. The composition of claim A1-A52, wherein said first peptoid is H-Ntridec-NLys-Nspe-Nspe-NLys-NH₂.

A58. The composition of claim A1-A52, wherein said first peptoid is H-(NLys-Nspe-Nspe) ₃-NLys-Nspe-NH₂.

A59. The composition of claim A1-A52, wherein said first peptoid is H-(NLys-Nspe-Nspe) ₂-NH₂.

A60. The composition of claim A1-A52, wherein said first peptoid is H-Ndec-(NLys-Nspe-Nspe) ₂-NH₂.

A61. The composition of claim A1-A52, wherein said first peptoid is H-Ndec-(NLys-Nspe-Nspe (p-Br))₂-NH₂.

A62. The composition of claim A1-A52, wherein said first peptoid is H-Ntridec-(NLys-Nspe-Nspe (p-Br))₂-NH₂.

A63. A method for treating a subject, comprising:
diagnosing the subject as having cancer; and
treating the subject with a pharmaceutically acceptable amount of the composition of claims A1-A62.

A64. A method for treating a subject, comprising:
diagnosing the subject as having a viral infection; and
treating the subject with a pharmaceutically acceptable amount of the composition of claims A1-A62.

A65. A method for treating a subject, comprising:
diagnosing the subject as having a fungal infection; and
treating the subject with a pharmaceutically acceptable amount of the composition of claims A1-A62.

A66. A method for treating a subject, comprising:
diagnosing the subject as having a bacterial infection; and
treating the subject with a pharmaceutically acceptable amount of the composition of claims A1-A62.

A67. A method for treating a subject, comprising:
diagnosing the subject as having a parasitic infection; and
treating the subject with a pharmaceutically acceptable amount of the composition of claims A1-A62.
